(19) United States
(12) Patent Application Publication
(10) Pub. No.: US 2016/0089412 A1
(43) Pub. Date: Mar. 31, 2016

(54) REMEDIAL TONIC

(71) Applicant: CHERI A. GALLAGHER, ST. LOUIS, MO (US)

(72) Inventor: CHERI A. GALLAGHER, ST. LOUIS, MO (US)

(21) Appl. No.: 14/497,545

(22) Filed: Sep. 26, 2014

(51) Int. Cl. A61K 36/886; A61K 36/53; A61K 36/63; A61K 36/185; A61K 36/28

(52) U.S. Cl. CPC A61K 36/886; A61K 36/185; A61K 36/28; A61K 36/63; A61K 36/53

(57) ABSTRACT

A medicinal tonic has a unique combination of ingredients. The tonic is composed, proportionally, of approximately 66 fluid ounces of organic Aloe juice, 60 ounces of organic Aloe gel, 16 grams be weight of wild lapacho bark extract, and 20 milliliters each of organic hyssop, organic or wild milk thistle extract and organic, naturally farmed olive leaf extract. The ingredients produce a new and medicinal compound that prevents or alleviates natural occurring diseases or conditions and helps heal wounds and other ailments and has other salutary effects on the human body.

### BACKGROUND OF THE INVENTION

This invention relates to the field of herbal remedies and medications. In particular, a novel and unique mixture of herbs and other naturally occurring substances is presented that has unusual and unexpected qualities and attributes.

The field of herbal extracts and natural cures has grown in the past decades. Herbal cures and remedies are now generally accepted as salutary substances for use by individuals for a variety of ailments and conditions. Some remedies are for specific diseases or maladies such as diabetes, allergies, prostate conditions or anemia. Other remedies are topical for use as sunscreen or to promote healing of wounds. It is an object of this invention to provide a unique herbal mixture that helps wounds, bruises and cuts to heal when used topically on the external skin.

Some herbal remedies may also be taken internally to promote health or well-being or to combat disease. These internally applied mixtures may have multiple good effects on the health, well-being or comfort of the individual. This particular composition boosts the immune system and is a general body tonic. It aids the body in fighting diabetes and cancer. It will help in deterring colds, the flu, sinus infections and allergies. If a person already has one or more of the above listed maladies, the tonic helps to relieve the symptoms and shorten the time required to overcome the ailment or disease. It is still another object of this invention to provide a tonic to be taken internally that will promote health and wellness.

Other and further objects of this new invention will become apparent upon reading the following description.

### BRIEF DESCRIPTION OF THE INVENTION

This invention comprises a unique combination of ingredients made in a new and novel way. Aloe Vera juice, Aloe Vera gel, hyssop herb extract, milk thistle seed extract, olive leaf extract and lapacho bark extract are combined to produce the product. The tonic thus produced is good for common ailments such as constipation and helps prevent or ameliorate the symptoms of many common diseases and conditions. The mixture may be taken internally by drinking or may be applied externally to the skin topically.

### DETAILED DESCRIPTION OF THE INVENTION

The Aloe Vera health mixture comprises Aloe Vera juice, Aloe Vera gel, hyssop herb extract, milk thistle seed extract, olive leaf extract and lapacho bark extract, in proportion, by a volume and weight as below described The tonic is produced using particular steps to create the new and novel composition.

In the preferred embodiment, a herbalist combines the component substances to manufacture one US gallon (128 ounces) of the tonic composition.

The herbalist starts with 66 fluid ounces of organic Aloe Juice (scientific name Aloe Barbadensis) and mixes the Aloe Juice with 60 fluid ounces of organic Aloe Gel (scientific name Aloe Barbadensis).

Next, 12 grams by weight of wild lapacho bark extract (scientific name Tabetuia Avellaneda) are added to the Aloe Vera mixture.

Finally, 20 ml by volume of organic hyssop extract (scientific name Hyssopis officinalis), and 20 ml by volume of organic or wild milk thistle seed extract (scientific name Silybum marianum) and 20 ml by volume of organic or naturally farmed olive leaf extract (scientific name Oleo europaea) are added to the mixture. The composition is then shaken until all the ingredients have been dissolved into one tonic, liquid composition.

The combination of the above ingredients produces 128 fluid ounces by volume of the fluid tonic. This may be conveniently divided into 16 or 32 ounce bottles. The mixture described will thus yield four 32 ounce bottles or eight 16 ounce bottles for use.

The ingredients for the tonic herein are set out as fluid ounces, milliliters by volume or by weight in grams to make 128 ounces of the composition. However, it is to be understood that this description is in proportion so that, for example, one-half of the tonic described (64 ounces) would require one-half of the listed ingredients.

It is recommended that the tonic be applied externally or taken internally 1 to 2 times per day and that 1 to 2 ounces be used per application or dose.

It is to be noted that the measure of the above ingredients is for information purposes only and are not means as a limitation on the exact composition of the tonic. After much experimentation, it has been found that ingredient variances of plus or minus 5% by volume of the Aloe Vera juice and Aloe Vera gel, are still within the keeping and disclosure of this invention. Further, plus or minus 2 percent by volume of the hyssop extract, milk thistle extract and olive leaf extract are still within the spirit and disclosure of this invention. The wild lapacho extract powder additive can have a range of plus or minus 3 percent while still keeping within the spirit of this disclosure.

Having fully disclosed my invention, I claim:

1: A medicinal tonic to be applied externally or taken internally, comprising, in proportion:
(a) Sixty-six (66) fluid ounces of Aloe juice; combined with
(b) Sixty (60) fluid ounces of Aloe gel; combined with
(c) Twelve (12) grams by weight of lapacho bark extract; combined with
(d) Twenty (20) milliliters of hyssop, twenty (20) milliliters of milk thistle extract and twenty (20) milliliters of olive leaf extract.

2: A medicinal tonic to be applied externally or taken internally, comprising, in proportion:
(a) Between 69.3 and 62.7 fluid ounces of Aloe juice; combined with
(b) Between 63 and 57 fluid ounces of Aloe gel; combined with
(c) Between 12.36 and 11.64 grams by weight of lapacho bark extract; combined with
(d) Between 20.4 and 19.6 milliliters by volume of hyssop, 20.4 and 19.6 milliliters by volume of milk thistle extract and 20.4 and 19.6 milliliters by volume of olive leaf extract.

3: A method of making a medicinal tonic to be applied externally or taken internally, comprising, the steps of:
(a) Taking between 69.3 and 62.7 fluid ounces of Aloe juice and combining said Aloe juice, in proportion, with
(b) Between 63 and 57 fluid ounces of Aloe gel; and combining said Aloe juice and Aloe gel mixture, in proportion, with
(c) Between 12.36 and 11.64 grams by weight of lapacho bark extract, in proportion, and combining said Aloe juice, Aloe gel and lapacho extract mixture with
(d) Between 20.4 and 19.6 milliliters by volume of hyssop, 20.4 and 19.6 milliliters by volume of milk thistle extract and 20.4 and 19.6 milliliters by volume of olive leaf extract, in proportion,
further comprising the step of shaking said mixture of ingredients until all of said ingredients are dissolved into a fluid tonic.
